(19) United States
(12) Patent Application Publication
(10) Pub. No.: US 2025/0050071 A1
(43) Pub. Date: Feb. 13, 2025

(54) INTRODUCER SYSTEM WITH INFLATABLE VALVE

(71) Applicant: Echo Medical, LLC, Irvine, CA (US)

(72) Inventors: Stephen Geist, Bend, OR (US); Alexander Siegel, Aliso Viejo, CA (US); Cooper R. Rickerson, Costa Mesa, CA (US); Belinko K. Matsuura, Encinitas, CA (US); Dalton W. Wilmers, San Diego, CA (US)

(73) Assignee: Echo Medical, LLC, Irvine, CA (US)

(21) Appl. No.: 18/801,382

(22) Filed: Aug. 12, 2024

Related U.S. Application Data
(60) Provisional application No. 63/519,110, filed on Aug. 11, 2023.

(51) Int. Cl. A61M 25/06; A61M 29/00; A61M 39/06

(52) U.S. Cl. CPC A61M 25/0662; A61M 29/00; A61M 39/0613; A61M 2039/0626; A61M 2039/064; A61M 2039/0673; A61M 2039/0686; A61M 2205/0216; A61M 2205/3327; A61M 2205/3331; A61M 2205/583; A61M 2209/00

(57) ABSTRACT

The present invention discloses an introducer system comprising an introducer sheath, a valve assembly, and a medical device passage. The valve assembly includes a flexible material with an inner surface exposed along a portion of the medical device passage and an outer surface defining a valve membrane chamber. Additionally, a compliance chamber within the valve assembly is in communication with the valve membrane chamber and houses a piston assembly movable between positions to accommodate inflation media.

### RELATED APPLICATIONS

This application claims benefit of and priority to U.S. Provisional Application Ser. No. 63/519,110 filed Aug. 11, 2023 entitled Introducer System with Inflatable Valve, which is hereby incorporated herein by reference in its entirety.

### BACKGROUND

Introducer sheaths are commonly used to introduce guidewires, catheters, and similar devices into the vascular system of a patient. A valve is also typically used with an introducer sheath to maintain hemostasis and prevent excessive bleeding. Such hemostasis valves typically remain closed when no instruments are positioned within the introducer sheath and seal around any instrument when positioned within the introducer sheath.

### SUMMARY

In some aspects, the techniques described herein relate to an introducer system, including: an introducer sheath; a valve assembly connected to the introducer sheath; a medical device passage defined by the introducer sheath and the valve assembly; a flexible material within the valve assembly having an inner surface exposed along a portion of the medical device passage and an outer surface at least partially defining a valve membrane chamber; and, a compliance chamber within the valve assembly and in communication with the valve membrane chamber; the compliance chamber further including a piston assembly within the compliance chamber and movable between a plurality of positions to accommodate inflation media.

In some aspects, the techniques described herein relate to an introducer system, wherein the piston assembly includes a piston member connected to a piston seal and a biasing member, where the biasing member biases the piston member to reduce a working volume of the compliance chamber.

In some aspects, the techniques described herein relate to an introducer system, wherein the compliance chamber is positioned annularly around at least a portion of the valve membrane chamber.

In some aspects, the techniques described herein relate to an introducer system, wherein the piston member is a ring.

In some aspects, the techniques described herein relate to an introducer system, wherein the biasing member is a spring.

In some aspects, the techniques described herein relate to an introducer system, wherein the spring is a helical coil spring, a leaf spring, a disk spring, a flat spring, or a machined spring.

In some aspects, the techniques described herein relate to an introducer system, wherein the biasing member is a resilient compressible material, a pneumatic piston mechanism, or a hydraulic piston mechanism.

In some aspects, the techniques described herein relate to an introducer system, wherein the piston seal is a cup seal having an annular channel.

In some aspects, the techniques described herein relate to an introducer system, wherein the compliance chamber further includes an annular ridge positioned to mate with the annular channel of the cup seal.

In some aspects, the techniques described herein relate to an introducer system, wherein the flexible material has a generally tubular shape.

In some aspects, the techniques described herein relate to an introducer system, wherein the generally tubular shape has a flexible middle portion and radially enlarged end portions.

In some aspects, the techniques described herein relate to an introducer system, wherein the flexible material forms a plurality of longitudinal pleats extending radially outward.

In some aspects, the techniques described herein relate to an introducer system, wherein the generally tubular shape further includes a slit valve covering a lumen of the generally tubular shape.

In some aspects, the techniques described herein relate to an introducer system, further including a clamp connected to the valve assembly, and having a closed clamp position configured to clamp a proximal portion of a dilator and an open clamp position configured to release the proximal portion of the dilator and allow longitudinal movement of the dilator relative to the introducer sheath.

In some aspects, the techniques described herein relate to an introducer system, further including a pressure measurement device.

In some aspects, the techniques described herein relate to an introducer system, wherein the valve assembly further includes a valve housing where at least part of the valve housing is transparent, and further including indicia indicating a pressure value of inflation media within the valve assembly.

In some aspects, the techniques described herein relate to an introducer system, wherein the piston member, the piston seal, or both, are configured to align with the indicia to indicate the pressure value of the inflation media within the valve assembly.

In some aspects, the techniques described herein relate to an introducer system, wherein the pressure measurement device is an electronic pressure transducer and an electronic display electrically connected to the pressure transducer.

In some aspects, the techniques described herein relate to an introducer system, wherein the compliance chamber further includes a plurality of compliance chambers.

In some aspects, the techniques described herein relate to an introducer system, wherein the plurality of compliance chambers are arranged annularly around the medical device passage.

In some aspects, the techniques described herein relate to an introducer system, including: an introducer sheath; a valve assembly connected to the introducer sheath; a medical device passage defined by the introducer sheath and the valve assembly; a valve member having an inflated configuration sealing the medical device passage within the valve assembly; and, a piston assembly within the valve assembly and in communication with the valve member.

In some aspects, the techniques described herein relate to an introducer system, wherein the piston assembly is annularly positioned around the medical device passage.

In some aspects, the techniques described herein relate to an introducer system, including: a valve assembly connected to an introducer sheath; a medical device passage defined by the valve assembly; a valve member having an inflated configuration sealing the medical device passage within the valve assembly; and, a piston assembly within the valve assembly and in communication with the valve member.

In some aspects, the techniques described herein relate to an introducer system, further including an introducer sheath connected to the valve assembly and further defining the medical device passage.

In some aspects, the techniques described herein relate to an introducer system, including: an introducer sheath; a valve assembly connected to the introducer sheath; a medical device passage defined by the introducer sheath and the valve assembly; and, a pressure measurement mechanism configured to communicate a pressure within the valve assembly.

In some aspects, the techniques described herein relate to an introducer system, wherein the pressure measurement mechanism includes a transparent portion of a housing of the valve assembly and wherein the pressure measurement mechanism is configured to measure an inflation media within the valve assembly.

In some aspects, the techniques described herein relate to an introducer system, further including indicia located on the housing of the valve assembly; wherein the indica is configured to indicate a pressure of the inflation media within the valve assembly based on a position of components within a compliance chamber of the valve assembly.

In some aspects, the techniques described herein relate to an introducer system, wherein the pressure measurement mechanism includes a electronic pressure transducer connected to an electronic display configured to display a pressure value measured from the electronic pressure transducer.

In some aspects, the techniques described herein relate to a method of using an introducer system, including: providing an introducer system including an introducer sheath and a valve assembly, where a medical device passage is defined by the introducer sheath and the valve assembly; charging the valve assembly with inflation media until a flexible material is inflated to expand into and close the medical device passage; placing a medical device into the medical device passage; and, accommodating inflation media displaced by movement of the flexible material by the medical device via a compliance chamber within the valve assembly; wherein the compliance chamber including a piston assembly.

In some aspects, the techniques described herein relate to an introducer system, including: an introducer sheath; a valve assembly connected to the introducer sheath; a medical device passage defined by the introducer sheath and the valve assembly; and, a dilator; wherein the dilator includes a distal region having an annular channel; wherein the introducer sheath includes a distal end that decreases in diameter distally so as to fit within the annular channel of the dilator and minimize a transition between an outer surface of the dilator and an outer surface of the introducer sheath.

In some aspects, the techniques described herein relate to an introducer system, including: an introducer sheath; a valve assembly connected to the introducer sheath; a medical device passage defined by the introducer sheath and the valve assembly; and, a dilator; wherein the dilator includes a distal region having an annular channel; wherein the introducer sheath includes a distal end that decreases in diameter distally so as to fit within the annular channel of the dilator and minimize a transition between an outer surface of the dilator and an outer surface of the introducer sheath. 31. An introducer system, including: an introducer sheath; a valve assembly connected to the introducer sheath; a medical device passage defined by the introducer sheath and the valve assembly; a dilator including a distal region having an annular channel; wherein the introducer sheath includes a distal end that decreases in diameter distally so as to fit within the annular channel of the dilator and minimize a transition between an outer surface of the dilator and an outer surface of the introducer sheath; and, a clamp assembly having an open state allowing longitudinal movement of the dilator relative to the valve assembly and the introducer sheath, and a clamped state preventing longitudinal movement of the dilator relative to the valve assembly and the introducer sheath.

In some aspects, the techniques described herein relate to the introducer system of 31, wherein the clamp assembly is attachable to a proximal portion of the valve assembly via an interlock mechanism.

In some aspects, the techniques described herein relate to a method of using an introducer system, including: providing an introducer system including an introducer sheath and a valve assembly, where a medical device passage is defined by the introducer sheath and the valve assembly; inserting a dilator into the valve assembly and introducer sheath; adjusting a longitudinal position of the dilator to align a channel on a distal portion of the dilator with a distal end of the introducer sheath; and, clamping a proximal portion of the dilator to the valve assembly to prevent longitudinal movement of the dilator relative to the valve assembly and the introducer sheath.

In some aspects, the techniques described herein relate to the method of 33, wherein the step of inserting the dilator into the valve assembly further includes connecting an interlock assembly of the clamp assembly positioned on the proximal portion of the dilator with the valve assembly.

In some aspects, the techniques described herein relate to an introducer system, including: an introducer sheath; a valve assembly connected to the introducer sheath; a medical device passage defined by the introducer sheath and the valve assembly; a dilator including a distal region having an annular channel; wherein the introducer sheath includes a distal end that decreases in diameter distally so as to fit within the annular channel of the dilator and minimize a transition between an outer surface of the dilator and an outer surface of the introducer sheath; and, a sheath movement assembly including an actuator that longitudinally moves the introducer sheath relative to the valve assembly and the dilator so that a user may align the annular channel with the distal end of the introducer sheath.

In some aspects, the techniques described herein relate to the introducer system of 35, wherein the actuator includes a thumbwheel, a slider, or a tubular actuator.

In some aspects, the techniques described herein relate to a method of using an introducer system, including: providing an introducer system including an introducer sheath and a valve assembly, where a medical device passage is defined by the introducer sheath and the valve assembly; inserting a dilator into the valve assembly and introducer sheath; adjusting a longitudinal position of the introducer sheath to align a channel on a distal portion of the dilator with a distal end of the introducer sheath.

In some aspects, the techniques described herein relate to the method of 37, wherein the step of inserting the dilator into the valve assembly further includes connecting an interlock assembly of the dilator on the proximal portion of the dilator with the valve assembly.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 illustrates a side view of an introducer system 100 according to some examples.

FIG. 2 illustrates a perspective view of the introducer system 100 of FIG. 1, according to some examples.

FIG. 3 illustrates a magnified side view of the valve assembly 102 according to some examples.

FIG. 4 illustrates a perspective end view of the valve assembly 102 of FIG. 3 according to some examples.

FIG. 5 illustrates a cross-sectional view of the valve assembly 102 according to some examples.

FIG. 6 illustrates a cross-sectional view of the valve assembly 102 according to some examples.

FIG. 7 illustrates a cross-sectional view of the valve assembly 102 according to some examples.

FIG. 8 illustrates a cross-sectional view of the valve assembly 102 according to some examples.

FIG. 9 illustrates a magnified cross-sectional view of a portion of the valve assembly 102 according to some examples.

FIG. 10 illustrates a side view of the valve membrane 128 in an inflated state according to some examples.

FIG. 11 illustrates a perspective view of the valve membrane 128 in an inflated state according to some examples.

FIG. 12 illustrates an end view of the valve membrane 128 in an inflated state according to some examples.

FIG. 13 illustrates a side view of the valve membrane 128 in a deflated state according to some examples.

FIG. 14 illustrates a perspective view of a spacer 136 according to some examples.

FIG. 15 illustrates a perspective view of a piston member 122 and piston seal 120 according to some examples.

FIG. 16 illustrates a cross-sectional view of a piston member 122 and piston seal 120 according to some examples.

FIG. 17 illustrates a side view of a piston member 122 and piston seal 120 according to some examples.

FIG. 18 illustrates a side perspective view of a valve assembly 102 with an outer housing 112 having a transparent portion 112A according to some examples.

FIG. 19 illustrates an enlarged cross-sectional view of a dilator 106 and introducer sheath 104 according to some examples.

FIG. 20 illustrates a magnified portion of Area 20 in FIG. 19 according to some examples.

FIG. 21 illustrates a perspective transparent view of valve housing 160 according to some examples.

FIG. 22 illustrates a cross-sectional view of a valve housing 160 with a plurality of compliance chambers 162 positioned at various annular locations around the medical device passage according to some examples.

FIG. 23 illustrates a side view of a an introducer system 180 in which a thumbwheel moves the introducer sheath 104 relative to the dilator 106.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

This specification is generally directed to an introducer system. Some examples of the present specification are directed to an introducer sheath, a valve assembly connected to the introducer sheath, and a passage defined by both components through which medical devices may be passed through. The valve assembly may comprise a flexible inflatable valve membrane that expands radially inward within the passage of the valve assembly to limit the escape of blood during use and maintain hemostasis within the patient. When a medical device, such as a dilator, guidewire, catheter, or other medical device is advanced through the passage of the valve assembly, the valve membrane maintains pressure against the medical device to maintain a seal.

The valve assembly may also include a compliance chamber that is in communication with a valve membrane chamber formed by the valve membrane. The compliance chamber helps accommodate (e.g., accept and push out) various amounts of inflation media needed within the valve membrane chamber to maintain the valve membrane in a state closing off the medical device passage when empty or sealing around a medical device if present. As a medical device enters the passage and moves the valve membrane radially outward, media inflating the valve membrane and valve membrane chamber is displaced into the media compliance chamber, which increases in size to accommodate the displaced media. If the medical device is removed, the media compliance chamber decreases in size to help inflate the valve membrane to close off the passage. Hence, the inflatable valve membrane may automatically seal around medical devices of a variety of different sizes, as well as when no devices are present in the passage.

In some examples, the compliance chamber may include a piston member that moves axially back and forth to change a working volume of the compliance chamber. As the piston member moves toward a first end of the compliance chamber, the working volume of the chamber (i.e., the area of the compliance chamber that contains inflation media) increases and greater amounts of inflation media may be accommodated. As the piston member moves toward a second end of the compliance chamber, the working volume of the chamber decreases and smaller amounts of inflation media may be accommodated. The piston member may be biased toward the second end to help maintain the valve membrane in an inflated state against itself or against a medical device if present.

In some examples, the introducer system may also comprise a dilator having a distal end with a proximally increasing conical taper and a length such that the taper of the distal end may extend out of a distal end of the introducer sheath. The dilator and introducer sheath both have features that may more gradually increase the radial transition between the two (i.e., there is less of an abrupt “step” at the transition between the two). In one example, the dilator may comprise a groove or channel that extends circumferentially around the dilator. The distal end portion of the introducer sheath may have a diameter that tapers in diameter, both internally and externally, in a distal direction. The taper of the introducer sheath may match that of the groove/channel of the dilator, thereby allowing at least the very distal end of the introducer sheath to have about the same diameter as portions of the dilator that are immediately distal to the groove/channel.

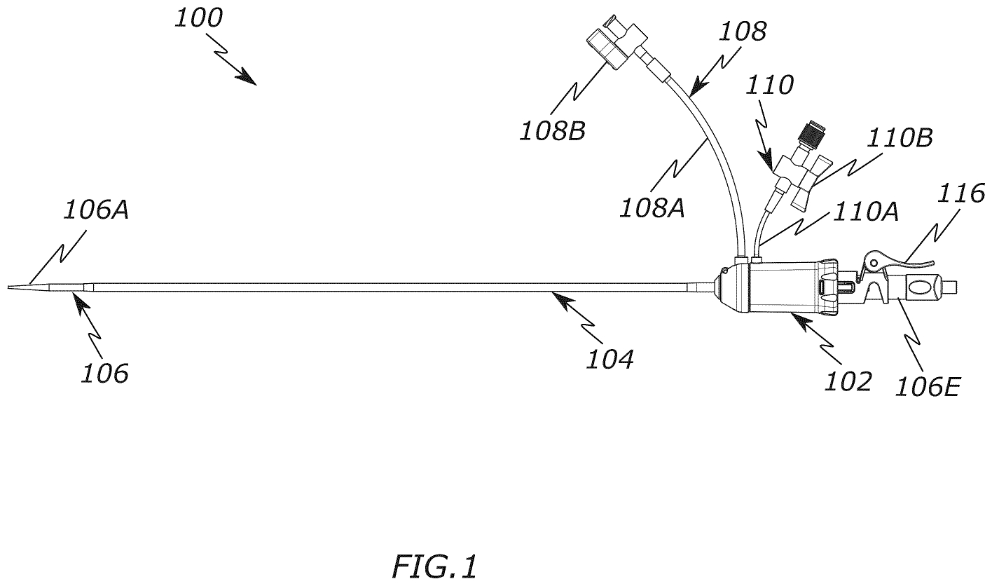

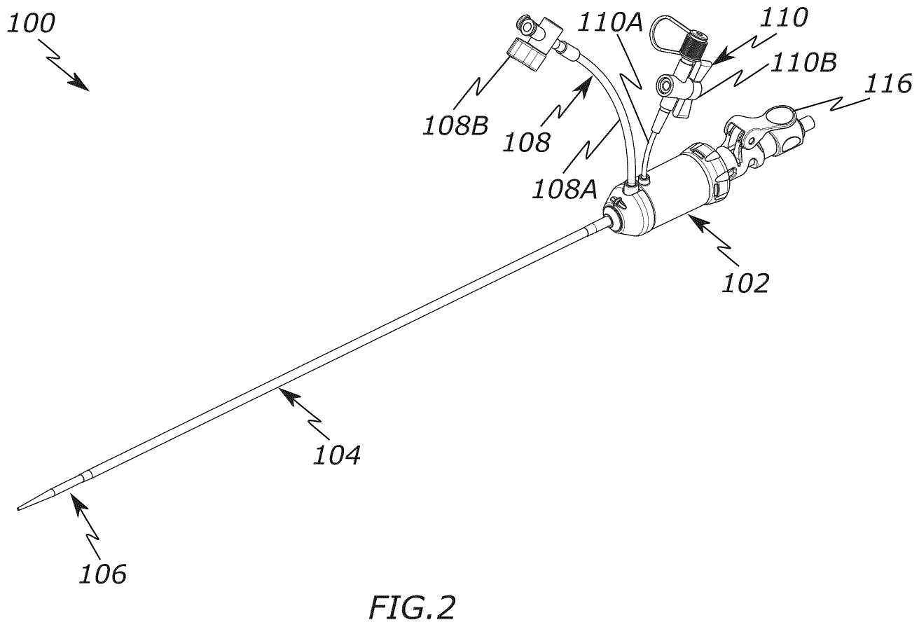

FIG. 1 illustrates a side view of one specific example of an introducer system 100 and FIG. 2 illustrates a perspective view of the introducer system 100, according to some examples. The introducer system 100 may comprise an introducer sheath 104 that is connected at its proximal end to a valve assembly 102 that is configured to maintain hemostasis when a distal portion of the introducer sheath 104 is positioned within a patient.

As described in further detail below, the introducer sheath 104 and the valve assembly 102 each have lumens extending between their proximal and distal ends that, together, form a unified medical device passage through which the dilator and other medical device may be advanced through. In other words, the valve assembly 102 comprises a lumen opening at its proximal and distal ends and the introducer sheath 104 also comprises a lumen opening at its proximal and distal ends, and together, these lumens form a single, unified medical device passage. Prior to placing the dilator 106 and introducer sheath 104 into the vasculature of a patient, a physician may charge or fill the valve assembly 102 with inflation media so that it limits blood leakage out of the patient and maintains hemostasis.

In some examples, the valve assembly 102 may be filled by injecting inflation media (e.g., fluid) into the inflation passage 110. The inflation passage may include an inflation tube 110A that opens into an interior of the valve assembly 102 at one end and that is attached to a valve 110B (e.g., a stopcock) at another end of the inflation tube 110A. The valve 110B may include a port into which a syringe or similar device may be connected to. After the inflation media has been injected or filled within the valve assembly 102, the valve 110B may be closed.

In some examples, the valve assembly 102 may also have a flush passage 108 through which saline or other liquids may be flushed into the passage of the introducer system 100 to remove air. The flush passage 108 may comprise a flush tube 108A that opens into the interior medical device lumen of the valve assembly 102 at its first end and connects to a valve 108B (e.g., a stopcock valve) at a second end of the flush tube 108A. Hence, saline or other liquids may be added to the passage to help remove air while the valve of the valve assembly 102 is closed.

The introducer system 100 may further include a dilator 106 having a conical tip 106A that is used to place at least a distal portion of the introducer sheath 104 within the patient. Once at least the distal portion of the introducer sheath 104 is positioned within the patient's vasculature, the dilator 106 may be removed from the introducer sheath 104 and valve assembly 102.

As discussed elsewhere in this specification, the introducer system 100 may include certain features of the introducer sheath 104 and dilator 106 that help minimize a transition size between outer surfaces of the two components and thereby reduce the likelihood of the introducer sheath 104 catching on certain features as it enters the patient (e.g., a channel on the dilator 106 and a tapered distal tip of the introducer sheath 104). Since these features of the introducer sheath 104 and dilator 106 are relatively small in longitudinal length, as well as their radial dimension, it may be difficult to reliably manufacture these components with the necessary tolerances for reliable alignment. Hence, the introducer system 100 may include a longitudinal alignment mechanism that allows a user to align the features of both components to the desired position to minimize any diameter transition.

In one example, the longitudinal alignment mechanism may comprise a clamp mechanism that locks the dilator 106 longitudinally in place relative to both the valve assembly 102 and the introducer sheath 104. In one example, the clamp mechanism may comprise a cam clamp mechanism that is engaged or disengaged via clamp lever 116 attached to proximal end portion 114. However, other known clamping mechanisms are also possible. Hence, the clamp mechanism allows the user to further adjust the longitudinal position of the features as necessary to achieve the minimized or no-step transition. In one example, the clamp mechanism may be releasably connected via an interlocking assembly (e.g., tabs/grooves, clips, mating features, etc.) to a proximal end of the valve assembly 102 such that the dilator can be attached and removed as needed. The clamp assembly may initially be located on a proximal portion of the dilator 106 (e.g., either non-removably or removably). In other examples, the clamp assembly may be non-removably fixed to the valve assembly 102.

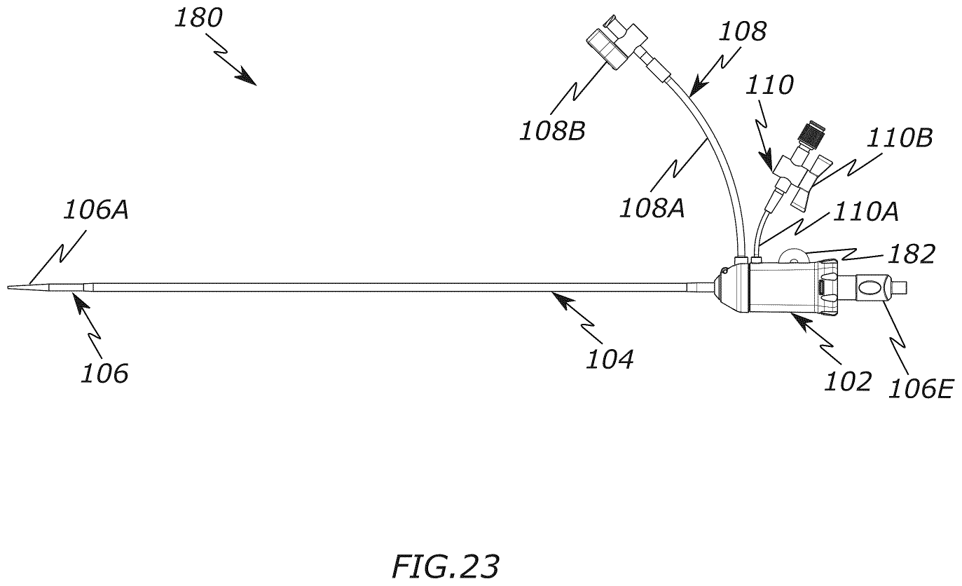

Alternatively, the introducer system may include a mechanism that allows the sheath 104 to move relative to the attached dilator 106 and the valve assembly 102. For example, FIG. 23 illustrates an introducer system 180 that is generally similar to the introducer system 100 except that, instead of a clamping mechanism, the valve assembly 102 may include a mechanism that longitudinally moves the introducer sheath 104. In the present example, a thumbwheel 182 may engage a toothed rack that is connected to the introducer sheath 104. However, other mechanisms, such as a thumb slider or an outer tubular adjuster that rotates via a screw mechanism, are also possible. In such examples, the proximal portion 106E of the dilator 106 may comprise an interlock mechanism that engages a proximal end of the valve assembly 102.

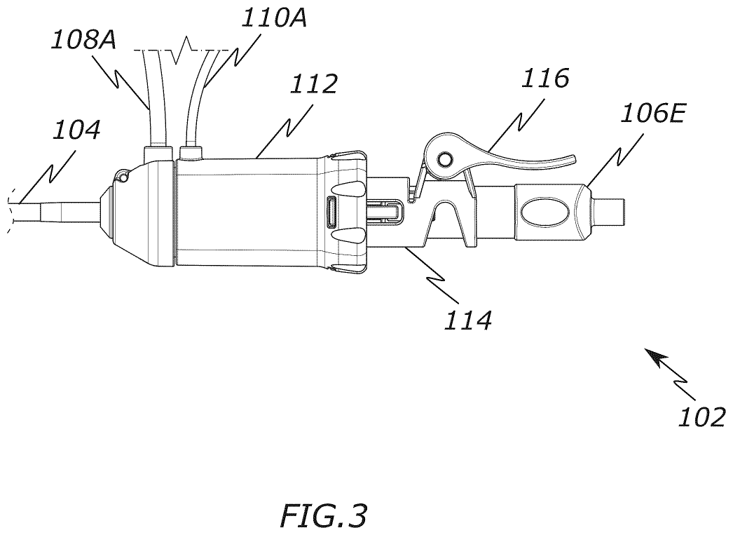

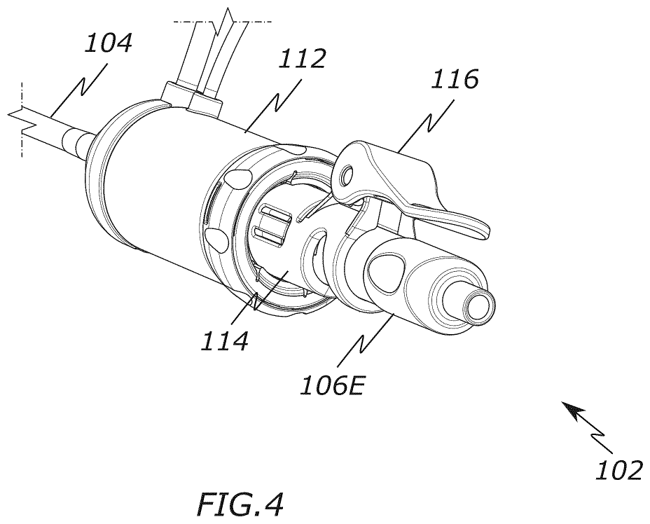

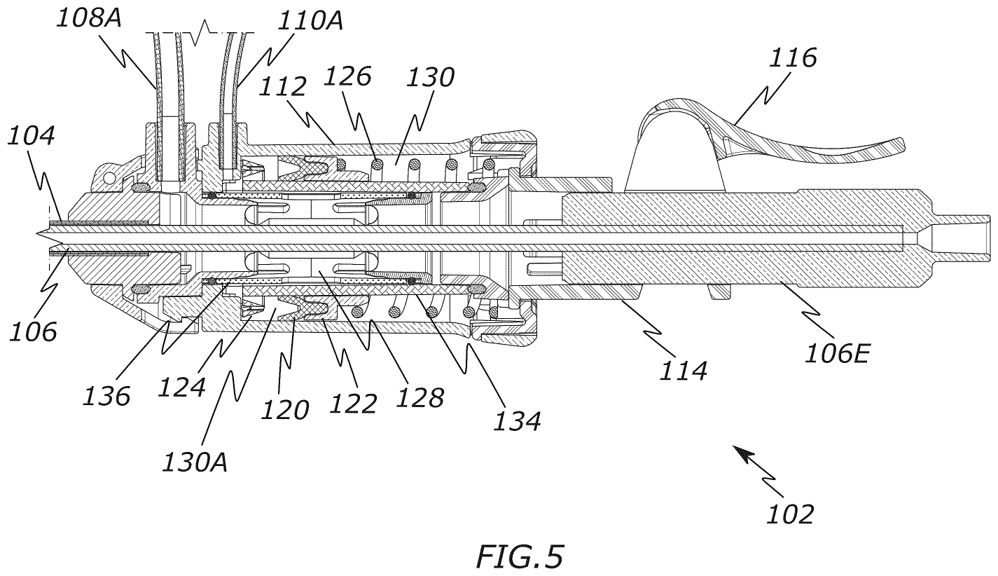

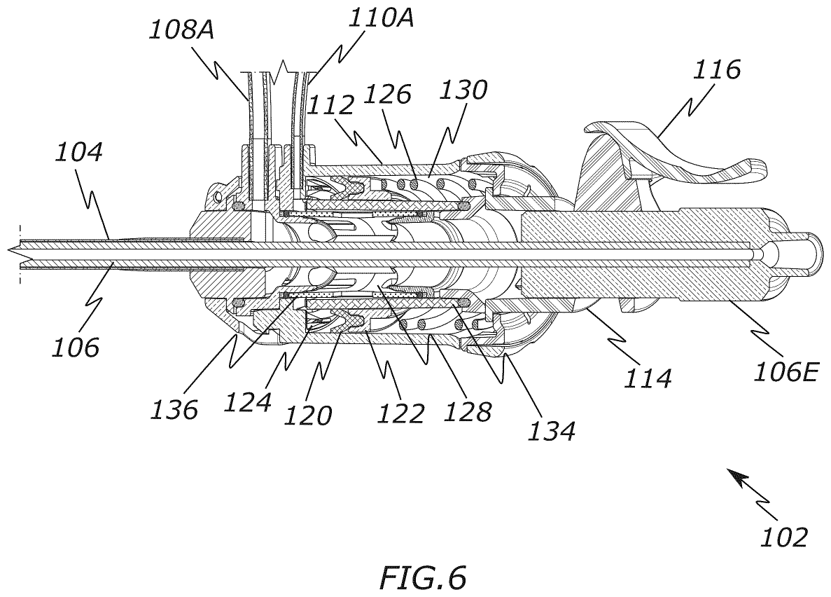

FIG. 3 illustrates a magnified side view of the valve assembly 102 and FIG. 4 illustrates a perspective end view of the valve assembly 102. FIGS. 5-9 illustrate various cross-sectional views of the valve assembly 102. All of these figures are discussed together below.

An outer housing 112 may form a shell containing many of the components of the valve assembly 102. In the present example, the outer housing 112 has a generally cylindrical shape, though noncylindrical shapes are also possible (e.g., rectangular).

In some examples, the valve mechanism of the valve assembly 102 may be comprised of at least two main components: a valve membrane 128 that inflates radially inwardly within an interior passage of the valve assembly 102 and a compliance chamber 130 that dynamically accommodates media displaced by movement of the valve membrane 128.

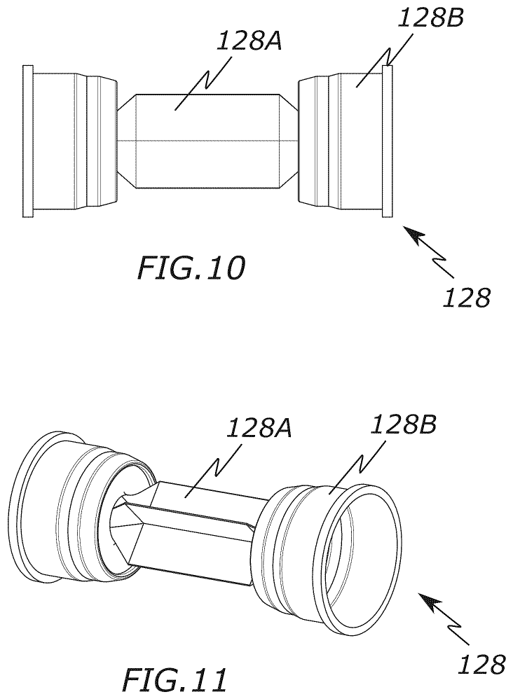

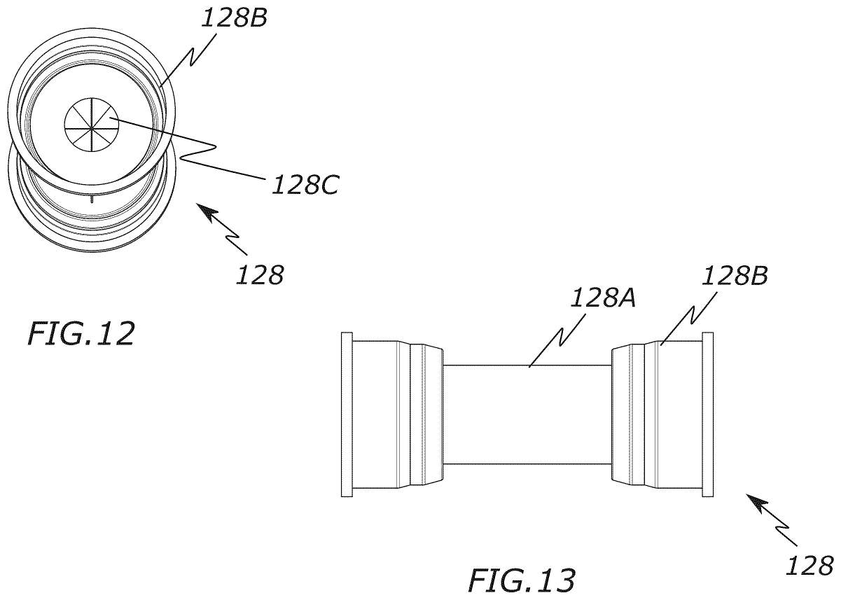

FIGS. 10-12 illustrate various views of one example of the valve membrane 128 in an inflated state and FIG. 13 illustrates the valve membrane 128 in a deflated state. The valve membrane 128 may have a generally tubular shape with enlarged end portions 128B that may be mounted within the outer housing 112 and a flexible middle portion 128A. A passage may extend longitudinally through the valve membrane 128 and one or both ends of the passage may optionally have slit valves 128C or similar types of valve mechanism.

In the “inflated” state seen in FIGS. 10-12, inflation media (e.g., fluid) presses on the outer surface of the flexible middle portion 128A which causes the flexible middle portion 128A to radially compress or distend inwards into its passage. When no medical devices, such as a dilator 106 or catheter, are positioned through the passage of the valve membrane 128, the inner surfaces of the flexible middle portion 128A contact each other, closing off the passage from blood. When one or more medical devices are positioned through the passage of the valve membrane 128, the flexible middle portion 128A contacts an outer surface of the one or more medical devices, sealing around the medical devices.

In the “deflated” state of FIG. 13, little or no inflation media presses against the outer surface of the flexible middle portion 128A and the passage of the valve membrane 128 may be partially or fully open. The state shown in FIG. 13 may be most likely to occur during an initial phase of use of the introducer system 100 when a user may remove air from the valve assembly 102 before adding inflation media (e.g., fluid) via the inflation passage 110. Hence, in such a “deflated” state, the flexible middle portion 128A may have its largest outward radial diameter but may otherwise be mostly or completely uninflated with inflation media (i.e., the valve membrane chamber 140 may be mostly/completely uninflated).

The valve membrane 128 may be composed of a noncompliant/nonelastic or partially noncompliant/nonelastic material, such as ePTFE, FEP, PFA, or FKM. In other examples, a knitted or woven fabric tube that has been coated to be impermeable to fluid and gas may also be used. In some examples, the valve membrane 128 may have a thickness within an inclusive range of about 20 to about 70 micron. Hence, in the “inflated” state of FIGS. 10 and 11, the flexible middle portion 128A is shown as having longitudinal pleats extending radially outward in the present example. However, the flexible middle portion 128A may form other shapes in the “inflated” state. The flexible middle portion 128A may have a circular cross-sectional shape, an oval cross-sectional shape, an hourglass shape with a smaller diameter near the middle, a reverse hourglass shape with a larger diameter near the middle, linear tapers in either proximal or distal directions, or similar variations.

While the flexible middle portion 128A of the present example is in the form of a generally tubular shape, other shapes and configurations are also possible. For example, the valve membrane 128 may instead comprise several discrete membranes positioned at radial/circumferential locations similar to the flexible middle portion 128A of FIGS. 10 and 11. Alternatively, the valve membrane 128 may instead be composed of a plurality of flexible tubes that each form balloon-like compartments that may also be positioned at radial/circumferential locations.

As previously noted, the valve assembly 102 may be charged or filled with liquid inflation media. Since liquid tends to be relatively noncompressible, placing a medical device through the inflated flexible middle portion 128A causes some of the inflation media to be displaced. The compliance chamber 130 provides a space for such inflation media by providing a dynamic working volume 130A that accepts inflation medial when needed (e.g., when a medical device is inserted) and pushes out inflation media to maintain the compliance or desired inflation level of the flexible middle portion 128A (e.g., when a medical device is removed from the flexible middle portion 128A). The working volume 130A is generally defined as the volume of the compliance chamber 130 that contains or is sized to contain inflation media at any given time and that may dynamically change in volume.

The compliance chamber 130 may take a variety of different shapes and positions within the valve assembly 102. In some examples, such as those in FIGS. 5-9, the compliance chamber 130 may be a generally cylindrical shape with an annular or ring-shaped cross section such that the compliance chamber 130 is positioned around the passage and valve membrane 128. Note, the annular cross section refers to a cross section taken generally perpendicular to an axis extending longitudinally through the medical device lumen of the valve assembly 102. The compliance chamber 130 may extend continuously around in a cross-sectional ring shape (e.g., an “O” shape) or may extend only partially cross sectionally around (e.g., a “C” shape). In the present example, the compliance chamber 130 is at least partially formed by an inner surface of the outer housing 112 and an inner tube 134.

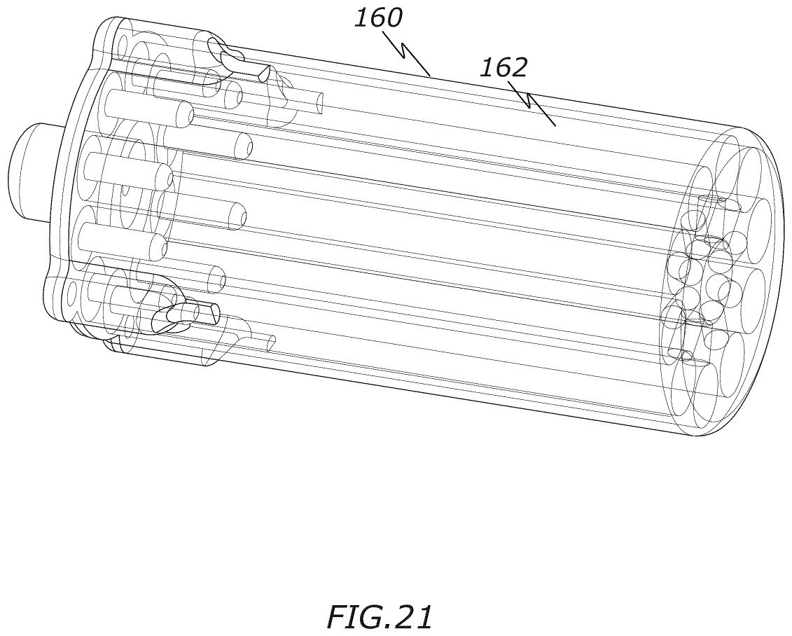

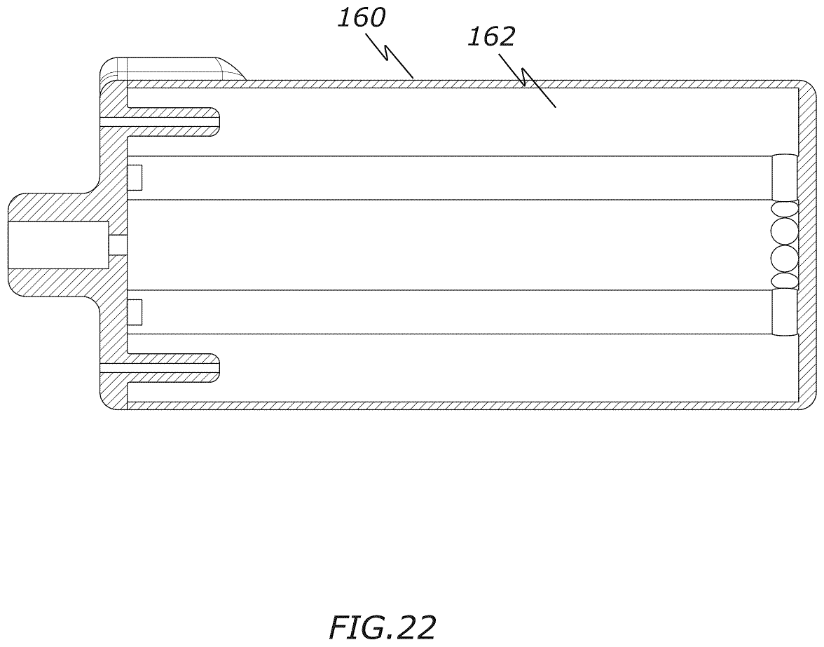

In other examples, the compliance chamber 130 may instead comprise a plurality of discrete chambers. For example, FIG. 21 illustrates a perspective and FIG. 22 illustrates a cross-sectional view of a valve housing 160 with a plurality of compliance chambers 162 positioned at various annular locations around the medical device passage, similar to a revolver. Each of these plurality of compliance chambers 162 may generally function similar to the annular compliance chamber 130 but with a circular cross sectional shape that forms a cylinder.

The compliance chamber 130 may dynamically change its working volume 130A via several different mechanisms. For example, the compliance chamber 130 may include a biased piston mechanism or piston assembly, an elastic membrane that may deform under pressure, one or more elastic balloons that may inflate under pressure, or similar mechanisms.

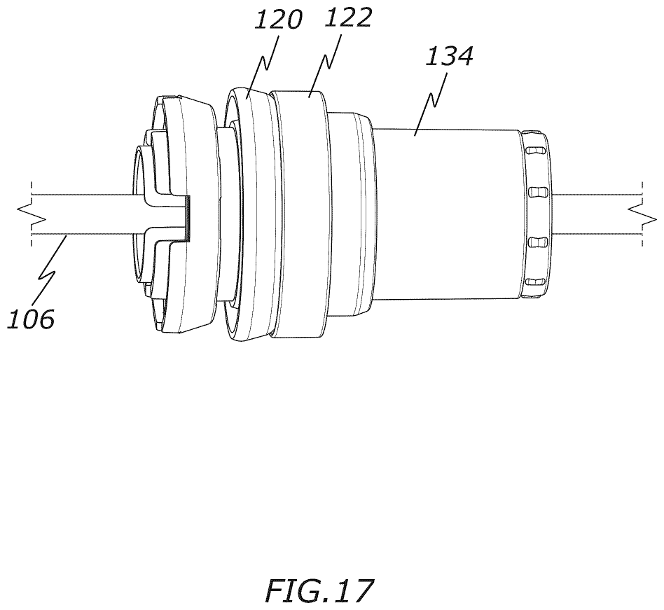

In the example of FIGS. 5-9, the compliance chamber 130 may include a piston mechanism comprising a piston member 122, a piston seal 120, and a spring 126. The piston member 122 and piston seal 120 are also shown alone in the perspective view of FIG. 15, the cross-sectional view of FIG. 16, and the side view of FIG. 17 in which the outer housing 112 has been removed.

The piston member 122 and piston seal 120 may be connected or attached to each other and may both be sized to fit within the compliance chamber 130 and move longitudinally between end regions of the compliance chamber 130. In the present example, both the piston member 122 and the piston seal 120 may have a generally ring shape.

The piston seal 120 may have a size and shape that seals the right/proximal portion (i.e., the working volume 130A) of the compliance chamber 130 from the left/distal portion of the compliance chamber 130. Specifically, the piston seal 120 may uniformly contact the inner surface of the outer housing 112 and the outer surface of the inner tube 134, creating the working volume 130A on the left/distal portion in which the inflation medial may enter.

In the present example, the piston seal 120 is a cup seal, having a channel 120A or a cross-sectional cup shape (e.g., a “V” or “C” cross sectional shape) that extends around its perimeter facing leftward/distally (e.g., an annular channel). The cup shape may exhibit a dynamic effect in that the V or C shape expands as pressure increases. In other words, higher pressure energizes the seal more. This can be important because the housing components may be injection molded and therefore may have draft along their length, so the annular gap increases in size from the base to the end. With a typical seal like an o-ring, a substantial amount of compression on the ring may occur at the base so that at the maximum point of travel the ring was still touching the inner and outer surfaces. In some cases, such excessive compression could be undesirable, depending on the manufacturing tolerances. Alternatively, the piston seal 120 may have a solid cross-sectional shape, such as a square, rectangular, circular, rounded, triangular, or similar shape. In another alternative example, the piston seal 120 may be an “X ring” which has a generally “X” cross sectional shape in which each side of the cross section forms a “V” or “C” shaped cup. The piston seal 120 may be composed of a flexible, resilient material.

The piston member 122 may be composed of a generally rigid material and have a generally annular/ring shape. In the present example, the piston member 122 may include a groove 122A into which a portion of the piston seal 120 is positioned within. The groove 122A may further include fastening features to further retain the piston seal 120, such as adhesive or mechanical anchors (e.g., barbs, lips, etc.).

The piston member 122 and piston seal 120 may be biased in a direction that reduces the size of the working volume 130A within the compliance chamber 130. In the present example, the piston member 122 and the piston seal 120 are biased towards the left/distal portion of the compliance chamber 130. In the present example, a spring 126 generates the biasing force and is located between the piston member 122 and one end (e.g., right/proximal end) of the compliance chamber 130. The spring 126 maybe a helical coil spring, as shown in the figures, or may be other types of springs, such as one or more leaf springs (e.g., a transversal leaf spring), disk springs, flat springs, machined springs, or similar variations. Alternatively, a resilient, compressible material may be used for a biasing force, such as a compressible polymer/foam. In another alternative example, the piston mechanism may be a pneumatic or hydraulic piston mechanism where gas or oil help provide a biasing force.

If the piston seal 120 comprises a cup seal with a channel 120A, the compliance chamber 130 may include a structural feature that helps maintain the “cup” shape if air is removed from the valve assembly 102. For example, if air is suctioned or vacuumed out of the valve assembly 102, the piston member 122 and piston seal 120 may be drawn to the left/distally until all air is removed and therefore a ridge 124 (e.g., an annular ridge with a mating shape to channel 120A) may be included around the left/distal end of the compliance chamber 130, allowing the channel 120A to engage with the ridge 124 and maintain its shape. This may help prevent portions of the piston seal 120 from folding over or otherwise losing their shape.

In the present example, the piston member 122 and the piston seal 120 move back and forth along an axis parallel to that of the medical device passage of the valve assembly 102, or between left/distal positions and right proximal positioned.

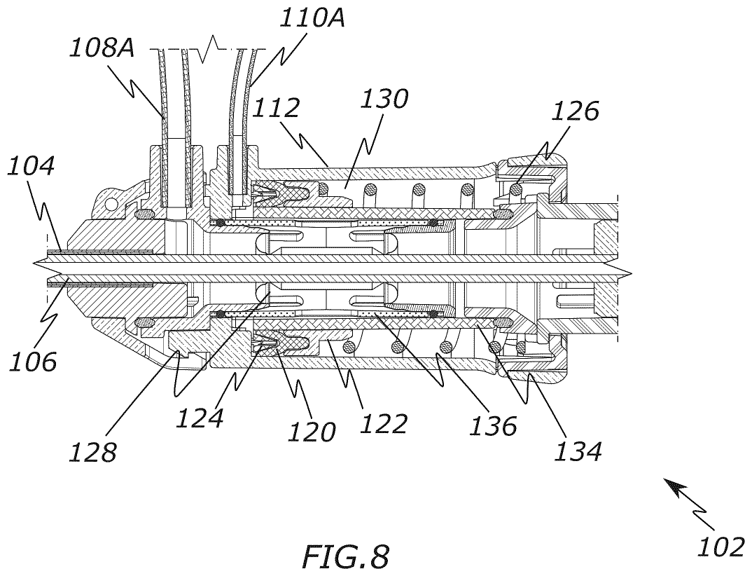

FIG. 8 illustrates the piston member 122 and the piston seal 120 in a leftward/distal position, which as previously discussed, may occur when the air is withdrawn from the valve assembly 102 prior to being charged/inflated with inflation media.

FIG. 5 illustrates the piston member 122 and the piston seal 120 in a position somewhat rightward/proximal from the distal end of the compliance chamber 130. While the dilator 106 is illustrated in the medical device passage of the valve assembly 102, this position of the piston member 122 and the piston seal 120 is more likely to occur when either a very small diameter medical device is positioned within the medical device passage of the valve assembly 102 or with no medical device present. Hence, much of the inflation media is located within the valve membrane chamber 140 created by the valve membrane 128.

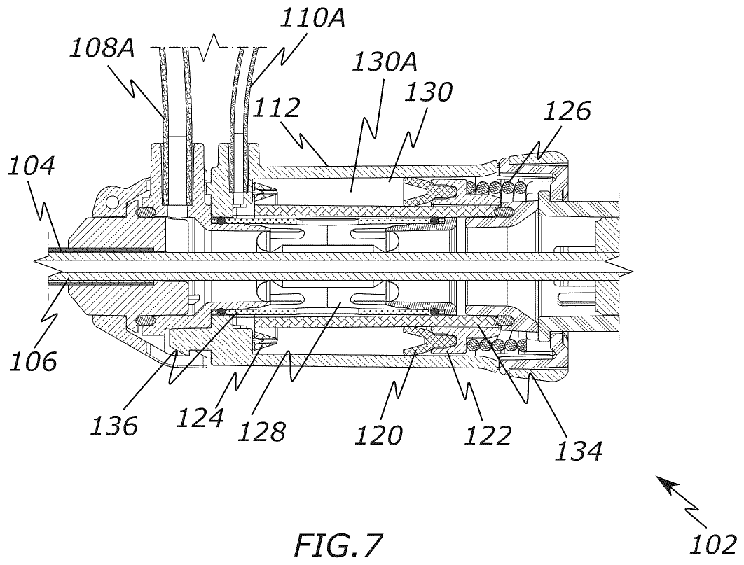

FIG. 7 illustrates the piston member 122 and the piston seal 120 in a position further rightward/proximal and near the proximal end of the compliance chamber 130. When relatively larger diameter medical devices are placed in the medical device passage of the valve assembly 102 and/or when multiple medical devices are placed side by side in the medical device passage of the valve assembly 102, some of the inflation media that was in the valve membrane chamber 140 created by the valve membrane 128 is displaced to the working volume 130A of the compliance chamber 130, moving the piston member 122 and piston seal 120 rightward/distally against the spring 126.

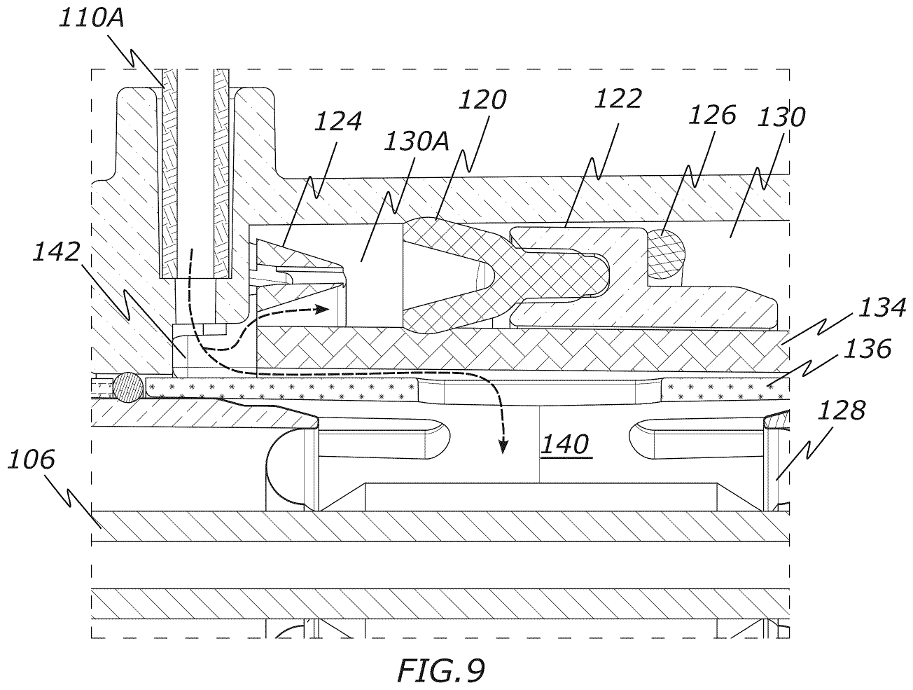

FIG. 9 illustrates a magnified view of a portion of the valve assembly 102 that better shows the path which the inflation media can take. While the arrows indicate an example direction of the inflation media in some circumstances, it should be understood that the inflation media may move back and forth during normal operation along portions of this path in either direction.

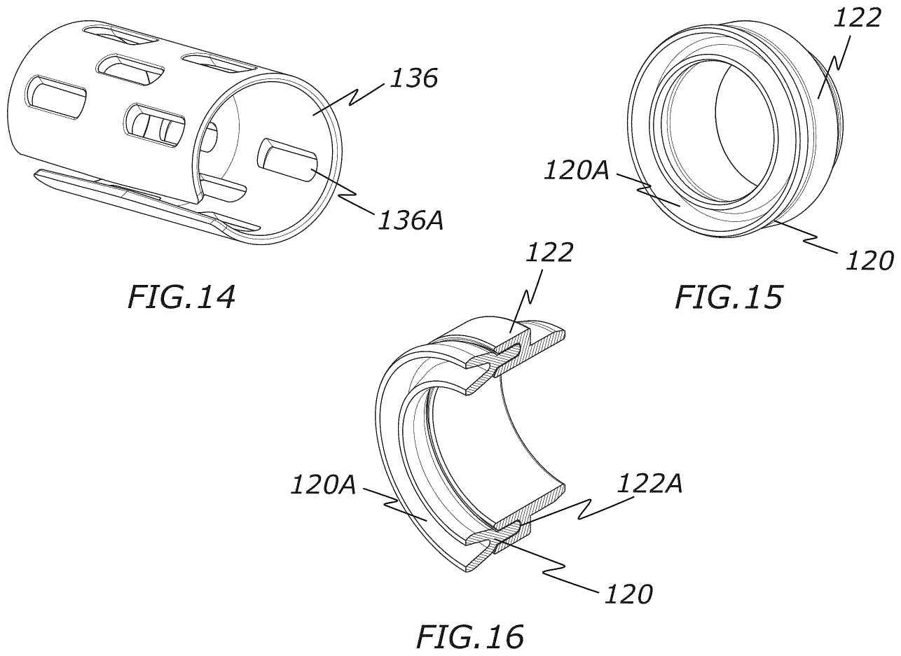

FIG. 9 also better illustrates the valve membrane chamber 140. In the present example, the valve membrane chamber 140 may be composed of the valve membrane 128, a spacer 136, and the inner tube 134. The spacer 136 may also be seen in FIG. 14 and may comprise a generally tubular shape with a plurality of apertures 136A to allow the inflation media to pass through. In some examples, the spacer 136 has an open or “C” cross sectional shape and in other examples the spacer 136 may have a closed or “O” cross sectional shape. As seen in FIG. 9, the spacer 136 may help create a longitudinal passage with the inner tube 134 that opens to an area 142. The area 142 also opens to the working volume 130A of the compliance chamber 130.

In this respect, inflation media may be charged or injected into the valve assembly 102 via inflation tube 110A, allowing the inflation media to pass into area 142 and then into valve membrane chamber 140 and the working volume 130A of compliance chamber 130. Then, as fluid is displaced from the valve membrane chamber 140, it enters the working volume 130A of the compliance chamber 130, pushing back the piston member 122 and piston seal 120 to increase the size of the working volume 130A as needed against the biasing force of the spring 126.

It may be desirable for the physician using the introducer system 100 to understand that enough inflation media has been introduced into the valve assembly 102 to sufficiently close the valve mechanism (e.g., valve membrane 128) of the medical device passage. In one example, this may be achieved by including a pressure measurement mechanism that measures the amount of pressure created by the spring 126 (or other biasing mechanism) against the inflation media.

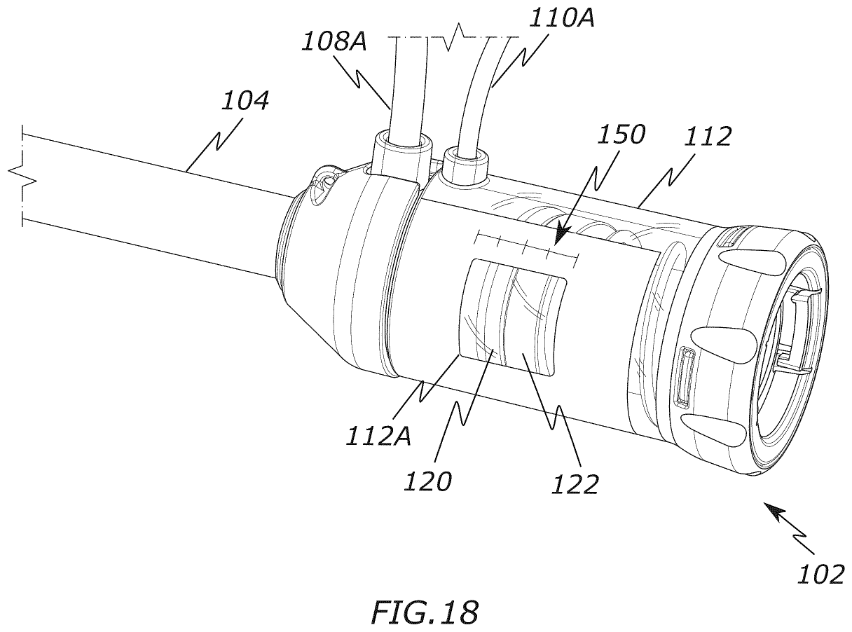

In one example, the pressure measurement mechanism may comprise an outer housing 112 that is partially or fully transparent to show the position of the piston member 122 and piston seal 120. For example, FIG. 18 illustrates a valve assembly 102 with an outer housing 112 having a transparent portion 112A. In some examples, the entire outer housing 112 may be composed of transparent material and in other examples, only a portion of the outer housing 112 may be composed of transparent material (e.g., a window or a tubular section).

Indicia 150 may also be included on or near the transparent portion 112A. These indicia 150 may be positioned and/or calibrated such that the position of the piston seal 120 and/or piston member 122 aligns with a specific pressure caused by the spring 126. In one example, the indicia 150 may include a numerical scale indicating a pressure measurement (e.g., PSI) and/or non-numerical markers that is arranged longitudinally along the outer housing 112 (e.g., parallel to an axis extending through the medical device passage). In some examples, the indicia 150 may at least include a range of values that include or extend beyond 11 PSI and 24 PSI (or their equivalents in other units). In some examples, it may be desirable to maintain the pressure of the inflation media within an inclusive range of about 6.5 PSI to about 8.5 PSI. For example, about 6.5, 7.0, 7.5, 8.0, or 8.5 PSI (plus-or-minus about 0.5 PSI). In some examples, a pressure of about 7.5 PSI may provide desirable sealing of the valve membrane 128.

Alternatively, a pressure measurement device external to the outer housing 112 and in communication with the interior of the valve assembly 102 may also be used.

In another alternative example, the indicia 150 may indicate a volume of the inflation media within the valve assembly 102.

In another alternative example, the indicia 150 may not indicate specific units of measurement and may instead provide a desired “position” or “zone of position” that the piston member 122 and/or the piston seal 120 should be positioned at during inflation. Such a position or zone may be calibrated to equate to a desired pressure within the valve assembly 102.

In another alternative example, the valve assembly 102 may include an electronic pressure transducer within the outer housing 112 that is positioned to measure a pressure of the inflation media. The pressure transducer may be connected to an electronic display mounted on the outside of the outer housing 112 or separate from the outer housing 112. The electronic display may display a pressure value and/or an indication that sufficient inflation media has been injected into the valve assembly 102.

In some examples, the dilator 106 and the introducer sheath 104 may include features that minimize a sizing transition between the dilator 106 and the distal portion of the introducer sheath 104. This may help the introducer sheath 104 from catching on the side walls of the vessels into which the introducer sheath is being inserted. The walls of these vessels often include calcific lesions which can restrict the sheath from advancing when an exposed edge of the sheath engages into them. Edges can also catch on prior implanted devices such as stents and filters.

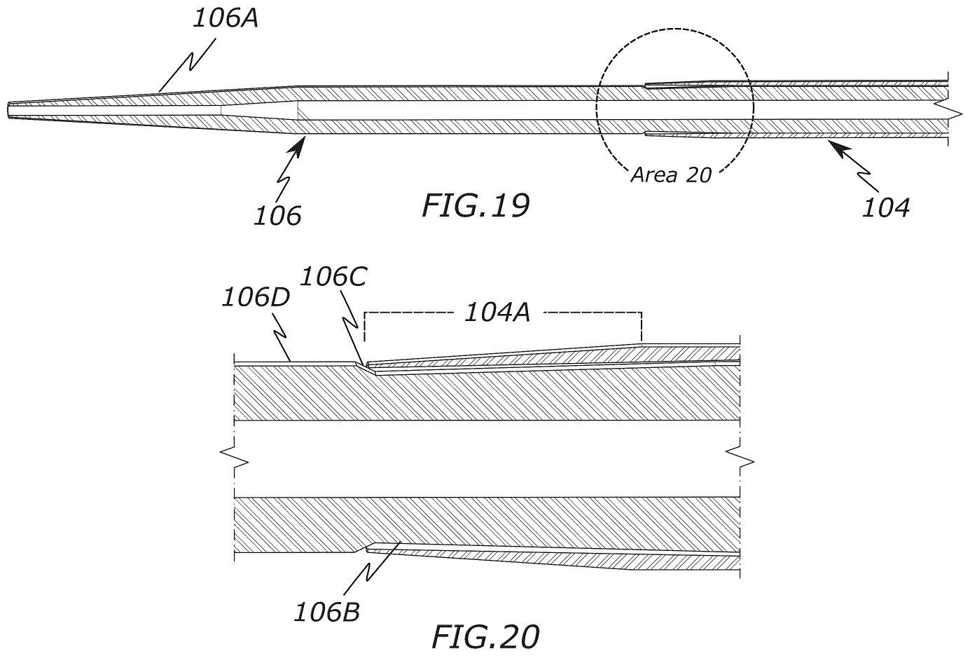

For example, FIG. 19 illustrates an enlarged cross-sectional view of the dilator 106 and introducer sheath 104, while FIG. 20 illustrates a magnified portion of Area 20 in FIG. 19. As previously discussed, the dilator may include a conical tip 106A that decreases in diameter in the distal direction to more easily be advanced into the vasculature of the patient. The dilator 106 may also include a region 106B that decreases in diameter proximally, followed by a short region 106C distal of the region 106B that increases in diameter distally. This may create a circumferential groove, channel, or indentation. A distal portion 104A of the introducer sheath 104 may also decrease in diameter distally such that, at least the tip of the introducer sheath 104 and possibly a small distal portion of the distal portion 104A, is generally uniform in its outer diameter with the portion 106D of the dilator 106 that is immediately distal of short region 106C. In that respect, the diameter between the portion 106D will be about the same as a distal end of the distal portion 104A. This may allow for a more uniform outer diameter transition between the dilator 106 and the introducer sheath 104.

In some examples, the axial angle of the distal portion 104A of the introducer sheath 104 matches or is about the same of the axial angle of the proximally-leading region 106B of the dilator 106. In some examples, the distal portion 104A of the introducer sheath 104 may have a length of about 0.05-0.25 inch and has an inner diameter that decreases from about 0.005 to about 0.030 inch (plus-or-minus about 0.001 inch) from the inner diameter of the lumen proximal of the distal portion 104. In some examples, the distal portion 104A of the introducer sheath 104 may be angled radially inwardly by about 3.633 degrees relative to an immediately proximal portion of the introducer sheath 104 with a constant diameter.

In some examples, the region 106B of the dilator 106 has a length of about 0.251 inch and the short region 106C has a length of about 0.014 inch (both plus or minus 0.003 inch). In some examples, the region 106B of the dilator 106 has an angle of about 1.6 degrees (plus-or-minus 1 degree) and the short region 106C has an angle of about 26 degrees (plus-or-minus 4 degrees), both radially inward and relative to the portion 106D.

The following is an example of a method of using an example introducer system 100 disclosed in this specification. However, variations of this method and performance of only parts of this method are also contemplated.

Generally, the introducer system 100 may be prepared for use. The valve 110B of the inflation passage 110 may be opened and the air may be removed from the valve assembly 102 (e.g., via syringe). The valve 110B may then be closed.

Next, a container of inflation media (e.g., a syringe) may be connected to the valve 110B and the valve 110B may be opened/actuated so that the inflation media is charged or injected into the valve assembly 102. The inflation media passes into the working volume 130A of the compliance chamber 130 and into the valve membrane chamber 140 formed by the valve membrane 128. The injection of inflation media is continued until a desired amount is placed into the valve assembly 102. In some examples, the operator may determine this amount of inflation media via indicia 150 and a position of the piston member 122 and/or the piston seal 120 (or alternatively one of the other previously described techniques). Once the desired amount of inflation media is injected into the valve assembly 102, the valve 110B may be closed. At this time, the medical device passage in the valve assembly 102 is in a closed position.

Saline or similar flushing fluid may be injected into valve 108B of the flush passage 108 so that the fluid may push out any air bubbles within the medical device passage of the valve assembly 102 and introducer sheath 104.

Next, the dilator 106 may be advanced into a proximal opening of the medical device passage of the valve assembly 102. In some examples, the clamp assembly may be a separate component already located on the end of the proximal end portion 106E of the dilator 106, and therefore is engaged via an interlock mechanism with the valve assembly 102 (e.g., tabs on a distal end of the clamp assembly that engage with grooves on a proximal face of the valve assembly 102).

Once the interlock mechanism is engaged, the axial/longitudinal position of the dilator 106 may be adjusted to align its groove (regions 106B and 106C) with reduced diameter portion of the distal portion 104A to create a uniform or “seamless” transition between the two components. The clamp lever 116 at the proximal end of the valve assembly 102 has an open position and a closed position that is actuated to clamp/release the proximal end portion 106E of the dilator 106 to the valve assembly 102, maintaining the longitudinal position of the dilator 106 and the introducer sheath 104 relative to each other. The clamp lever 116 may be engaged to lock the dilator 106 in its axial position.

The conical tip 106A and distal portion 104A of the introducer sheath 104 may have a guidewire lumen that is placed over a guidewire that has been previously advanced into the patient. The conical tip 106A and distal portion 104A of the introducer sheath 104 are advanced distally over the guidewire until the introducer sheath 104 is mostly within the vasculature of the patient. Since the valve membrane 128 is in an inflated state, it presses against and seals against the outer surface of the dilator 106 to substantially or mostly prevent blood leakage and maintain hemostasis.

The dilator 106 may be proximally withdrawing from the introducer sheath 104 and valve assembly 102. As the dilator 106 is removed, the inflated valve membrane 128 increases in size via pressure from the compliance chamber 130 and thereby closes the medical device passage.

Finally, one or more medical devices, such as a guidewire, catheter, etc., may be advanced into the valve assembly 102, into the introducer sheath 104, and into the patient. The valve membrane 128 displaces the inflation media into the compliance chamber 130 to accommodate the inserted medical device(s). A single device may be inserted into the introducer system 100 or two or more devices may be inserted side-by-side with each other.

In the examples of this specification, the introducer sheath 104 and the valve assembly 102 are fixed together in a non-removable manner. However, in other examples, the valve assembly 102 and the introducer sheath 104 may be separable.

In some examples, the inflation media may be saline, water, other fluids, foams, or gels. In other examples, the inflation media may be a gas, such as air. Typically, the use of fluids will be more desirable since it reduces the risk of a gas bubble entering the vasculature of the patient. However, a sufficiently sealed and/or isolated compliance chamber 130 and valve membrane chamber 140 may allow for the use of gases as inflation media.

While the compliance chamber 130 has been generally shown as being located within the outer housing 112 that also contains the valve membrane chamber 140, the compliance chamber 130 may alternatively be located in a separate housing connected with the outer housing 112 via a tube or similar structure.

While the present introducer system 100 has been described for use for accessing a patient's vasculature, other non-vasculature portions of a patient may also be accessed.

1. An introducer system, comprising:
an introducer sheath;
a valve assembly connected to the introducer sheath;
a medical device passage defined by the introducer sheath and the valve assembly;
a flexible material within the valve assembly having an inner surface exposed along a portion of the medical device passage and an outer surface at least partially defining a valve membrane chamber; and,
a compliance chamber within the valve assembly and in communication with the valve membrane chamber; the compliance chamber further comprising a piston assembly within the compliance chamber and movable between a plurality of positions to accommodate inflation media.

2. The introducer system of claim 1, wherein the piston assembly comprises a piston member connected to a piston seal and a biasing member, where the biasing member biases the piston member to reduce a working volume of the compliance chamber.

3. The introducer system of claim 2, wherein the compliance chamber is positioned annularly around at least a portion of the valve membrane chamber.

4. The introducer system of claim 3, wherein the piston member is a ring.

5. The introducer system of claim 2, wherein the biasing member is a spring.

6. The introducer system of claim 5, wherein the spring is a helical coil spring, a leaf spring, a disk spring, a flat spring, or a machined spring.

7. The introducer system of claim 2, wherein the biasing member is a resilient compressible material, a pneumatic piston mechanism, or a hydraulic piston mechanism.

8. The introducer system of claim 2, wherein the piston seal is a cup seal having an annular channel.

9. The introducer system of claim 8, wherein the compliance chamber further comprises an annular ridge positioned to mate with the annular channel of the cup seal.

10. The introducer system of claim 1, wherein the flexible material has a generally tubular shape.

11. The introducer system of claim 10, wherein the generally tubular shape has a flexible middle portion and radially enlarged end portions.

12. The introducer system of claim 10, wherein the flexible material forms a plurality of longitudinal pleats extending radially outward.

13. The introducer system of claim 10, wherein the generally tubular shape further comprises a slit valve covering a lumen of the generally tubular shape.

14. The introducer system of claim 1, further comprising a clamp connected to the valve assembly, and having a closed clamp position configured to clamp a proximal portion of a dilator and an open clamp position configured to release the proximal portion of the dilator and allow longitudinal movement of the dilator relative to the introducer sheath.

15. The introducer system of claim 1, further comprising a pressure measurement device.

16. The introducer system of claim 2, wherein the valve assembly further comprises a valve housing where at least part of the valve housing is transparent, and further comprising indicia indicating a pressure value of inflation media within the valve assembly.

17. The introducer system of claim 16, wherein the piston member, the piston seal, or both, are configured to align with the indicia to indicate the pressure value of the inflation media within the valve assembly.

18. The introducer system of claim 15, wherein the pressure measurement device is an electronic pressure transducer and an electronic display electrically connected to the pressure transducer.

19. The introducer system of claim 1, wherein the compliance chamber further comprises a plurality of compliance chambers.

20. The introducer system of claim 19, wherein the plurality of compliance chambers are arranged annularly around the medical device passage.

21-38. (canceled)
